(19) United States
(12) Patent Application Publication
(10) Pub. No.: US 2002/0127269 A1
(43) Pub. Date: Sep. 12, 2002

(54) MULTI-DOSE WOUND GEL

(76) Inventors: MICHAEL J. WARING, WIRRAL (GB); ELIZABETH JACQUES, HOOLE (GB)

Correspondence Address: T R FURMAN, BRISTOL MYERS SQUIBB COMPANY, 100 HEADQUARTERS PARK DRIVE, SKILLMAN, NJ, 08558

(21) Appl. No.: 09/341,821

(22) PCT Filed: Jan. 22, 1998
(86) PCT No.: PCT/EP98/00526

(30) Foreign Application Priority Data
Jan. 25, 1997 (GB) 9701552.3

(51) Int. Cl. A01N 25/34; A61K 9/70; A61F 13/00

(52) U.S. Cl. 424/443; 424/402

(57) ABSTRACT

A barrier aerosol vessel containing a wound gel for the treatment ol wounds. Wound gels packaged in this way have many advantages. Firstly, because there is positive pressure in the container, the vessel can be made to be self-sealing. This aids maintenance of product sterility. Secondly, it is possible to uise an aerosol using only one hand which makes application of the gel to the wound particularly easy. In the case of sinus wounds, where there is undermining of the tissue beneath the wound site and beyond its surface periphery, it is possible to insert the nozzle through the sinus to fill the cavity with gel. Thirdly, since the propellent is not mixed with the gel, the physical and chemical properties of the gel are not aflected by being dispensed in this manner.

This invention relates to a multi-dose wound gel. More particularly, this invention relates to a wound gel packaged in a multi-dose container, useful for treating wounds.

It is well known that the cleansing and debriding of wounds and the removal of wound exudate is important to the process of healing wounds. Commonly used wound dressings comprise gauze, foams, sponges, cotton wads or other fibrous materials. Gauze and other fibrous materials absorb fluids by capillary action with the disadvantage that when new tissue is formed as part of the healing process, it engulfs the fibres and is torn when the material is removed causing wound injury.

Various other materials have been used, such as gels hydrogels, granules and pastes to remove exudates from wounds. Certain wound gels are known to promote the healing of wounds. For instance they can keep the wound bed moist, cleanse the wound, debride necrotic matter by fluid donation and absorb exudate. Freshly generated tissue does not grow into the gel and thus injury on removal is avoided.

The gels are usually packaged in a tube and applied to the wound from the tube. The gels are usually in either a sterile or a preserved state. If packaged in a multi-dose tube there is a risk with some gels that once the tube is opened, bacteria will enter the tube and proliferate in the gel. For this reason some manufacturers include preservatives in the gel or package in single dose tubes. Some health care professionals are reluctant to introduce preservatives to a wound and so use single dose tubes containing sterile gel. This adds to the cost of the product and results in wastage if the whole contents of the tube is not used. There is therefore a need for a multi-dose gel packaged in such a way that contamination is minimised once the packaging is opened.

We have now found that is possible to package a gel in multi-dose packaging which minimises contamination once opened. This is achieved by the use of a barrier aerosol. Accordingly the invention provides a barrier aerosol vessel containing a wound gel for the treatment of wounds.

Aerosol barrier vessels are of the type where the product to be dispensed and the pressure generating media, ie the propellant, are maintained in isolation through separation on opposite sides of a barrier. This has many advantages in the context of wound gels. Firstly, because there is positive pressure in the container, the vessel can be made to be self-sealing. This aids maintenance of product sterility. Secondly, it is possible to use an aerosol using only one hand which makes application of the gel to the wound particularly easy. In the case of sinus wounds, where there is undermining of the tissue beneath the wound site and beyond its surface periphery, it is possible to insert the nozzle through the sinus to fill the cavity with gel. Thirdly, since the propellent is not mixed with the gel, the physical and chemical properties of the gel are not affected by being dispensed in this manner. It is therefore possible to use known gels which have a long clinical history with the advantages of being dispensed under pressure as described above.

Three main variants of barrier vessel exist. In a piston-type barrier vessel the barrier is a piston-like component that is mounted in the container in sliding relation to the inside surface of the container. The product to be dispensed is disposed on the valved side of the piston and the propellant, which generates pressure within the container, is on the opposite side of the piston. Examples of piston-type barrier packs are described in U.S. Pat. Nos. 3,033,923, 3,756,476 and 3,929,132.

In a second variant of an aerosol barrier vessel, a flexible collapsible inner container is affixed within an outer container opening either to the aerosol discharge valve or to the bead of the container opening. Patents which illustrate a barrier vessel of this variant are described in U.S. Pat. Nos. 3,788,521, 3,896,970 and 4,067,499.

In a third variant the barrier vessel is an unfolding cup-shaped barrier wherein the barrier has an outer wall terminating in a sealing flange, said outer wall being disposed contiguous to the inner wall of the container. The inner wall of the barrier is initially folded within the outer wall, the inner wall terminating in an end closing portion. The barrier is contained in a valved aerosol container and sealed at the joint formed between the sidewall and the bottom end closure of the container. Product is admitted through the valved opening of the container and propellant through a port in the bottom end closure of the container. Actuation of the valve reduces the pressure in the product compartment and results in the inner wall of the barrier unfolding from within the outer wall of the barrier and causing the end-closing portion of the inner wall of the barrier to advance and thereby urge the product toward the discharge port. This type of barrier vessel is illustrated in U.S. Pat. No. 3,109,463 and WO 96/02439.

The barrier aerosol vessel preferably used in the present invention is of the second or third type and comprises an inner container which contains the gel sealed by an opening valve with a discharge port for discharging the gel, an outer casing container covering the inner container and a pressure medium interposed between the inner container and the outer casing container. The use of this type of container enables the inner container to be filled with non-sterile gel while assembled in the outer casing container, sealed by the valve and then sterilised by steam sterilisation. The pressure medium can then be introduced without compromising the sterility of the, product. If a non-barrier type of aerosol were used then sterilisation would not be possible due to the presence of propellant in the wound gel.

Preferably the inner container is made of a thin flexible material such as plastic or metal foil, although metal foil is especially preferred to maintain sterility if a sterile gel is used. The outer casing container is also preferably metal such as aluminium which is pressure resistant. The outer container is preferably formed by compression moulding, thermoforming or the like, the inside provided with an inner protective coating and primed and the base provided with a valve to enable the container to be pressurised once the inner container has been filled and sealed. The inner container is preferably sealed by a valve which comprises a cup and a discharge port and closes off the outer container.

A barrier vessel suitable for use in the present invention is illustrated in the following figures:

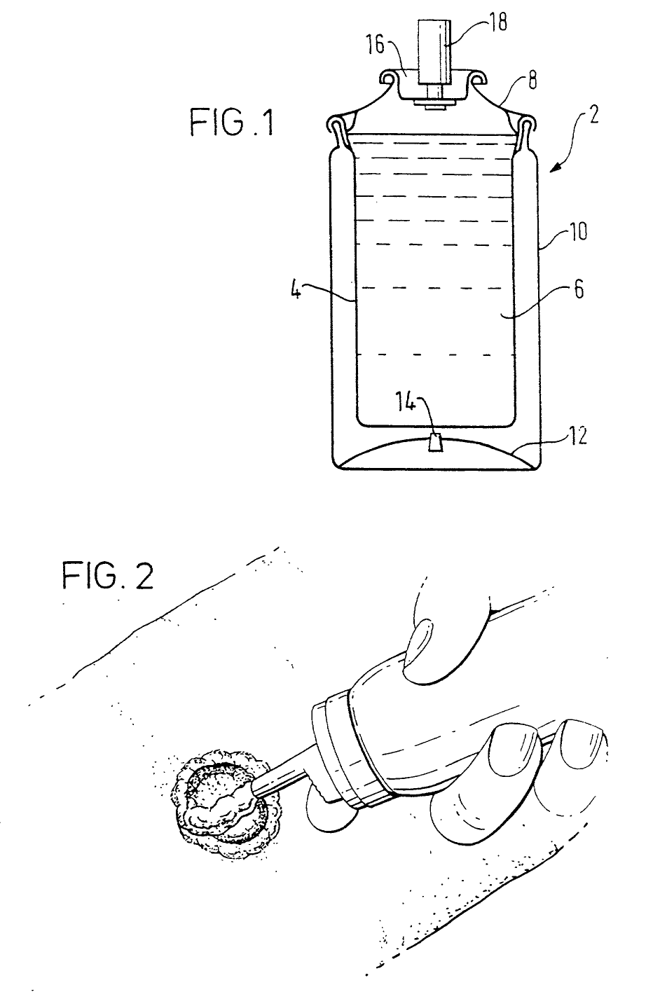

FIG. 1 is an elevation in section of one embodiment of the invention.

FIG. 2 is a perspective view of the vessel of the invention in use.

An example of a barrier aerosol vessel (2) suitable for use in the present invention is shown in FIG. 1 and comprises an inner container (4) which contains a gel (6) sealed by an opening valve (8) for discharging the gel (6), an outer casing container (10) covering the inner container (4) and a pressure medium (12) interposed between the inner container (4) and the outer casing container (10). The outer casing container is provided with a sealable port (14) to enable the pressure medium (12) to be introduced. The opening valve (8) comprises a cup (16) and discharge port (18). The whole of the opening valve (8), including cup (16) and port (18) will conventionally be covered with an applicator (not shown). Depression of which by the user causes the gel to exit the port (18) into a conventional nozzle or an extension nozzle depending on the use. Such nozzles can be separately packaged for single use.

FIG. 2 shows the aerosol vessel of the invention in use. In this view an applicator has been placed on the opening valve to aid application of the gel to a wound.

When the gel is to be dispensed the valve (8) is actuated, the pressure medium acts to collapse the inner container (4) and gel (6) flows from the discharge port (18) of valve (8).

The gel for use in the present invention is preferably a hydrocolloid gel and comprises a cellulose derivative, water and a polyol component. Such gels are described in EP-A-576523. More preferred is a gel comprising:

(a) optionally from about 0.05% to 10% by weight of a natural gelling agent;

(b) from about 1.0% to 106 by weight of a hydrocolloid;

(c) from about 5.06 to 30.0% by weight of a glycol and

(d) at least 50% by weight of water.

The most preferred composition contains 0.1% pectin, 3.4% sodium carboxymethyl cellulose, 15% propylene glycol and 81.5% water. Such gels are described in EP-A-567311 and EP-A-666081.

The natural gelling agent is preferably selected from pectin, alginic acid and salts thereof, carageenan, tragacanth, acacia, locust bean gum, guar gum, starch, agar and gelatin. More preferably the pectin is pectin with a high ester content derived from citrus peel consisting chiefly of the partial methyl esters of polygalachronic acid (approximately 65% of the carboxyl groups are esterified). Representatives of the pectin useful in the gel composition is that marketed under the name GENU pectin type VIS-L by Copenhagen Pectin. The natural gelling agent is preferably present in an amount from 0.05% to 1.00 by weight.

The hydrocolloid is preferably selected from sodium carboxymethyl cellulose, methyl cellulose, hydroxypropylmethyl cellulose, hydroxyethyl cellulose, hydroxymethylcellulose, hydroxypropyl cellulose, carboxyvinyl polymer and salts thereof, poloxamer for example Pluronic F127, xanthan gum, povidone, modified starches, and guar derivatives. The carboxymethyl cellulose is preferably sodium carboxymethyl cellulose present in an amount from about 2.0% to 4.5% by weight. The preferred sodium carboxymethyl cellulose is a high viscosity sodium carboxymethyl cellulose (typically in the range 2000-4500 cps as measured by Brookfield LV Viscometry of a lo solution, oven dry basis, 25° C. and spindle 4/30 rpm.

The glycol can be an aryl or alkylene glycol, preferably selected form the group of glycerol, polyethylene glycol, panthanol, and sorbitol. If the glycol is an alkylene glycol it is preferably propylene glycol present at from about 10.0% to 20.0% by weight.

The water used in the gel is preferably purified and pyrogen tree water and is present in an amount sufficient to bring the total composition up to 100% by weight.

Various optional ingredients can also be included in the gel composition such as preservatives eg, methylhydroxybenzoate and propylhydroxybenzoate. In addition, the wound gel composition can, if desired, contain small amounts (effective amounts) i.e. less than 5%, of pharmacologically active ingredients. For example, an antibiotic or antimicrobial agent such as, metronidazole, silver sulphadiazine, neomycin or penicillin, and antiseptic agent such as povidone iodine, and anti-inflammatory agent such as hydrocortisone or triamcinolone acetonide, or a skin protective agent such a zinc oxide can be included.

We have found that the invention performs particularly well if the gel has a viscosity of from 150 to 800 Pas as determined by Viscolog model MRV8 viscometer and a helical drive unit and PD spindle rotating at 2.5 rpm. Gels packaged in this way have been found to have particularly homogeneous viscosity due to the uniformity of the package compared to that found in tubes or other less symmetrical dispensing devices.

The barrier vessel containing a wound gel of the invention may be made by the method described in EP 0418724 or EP 0017147 to Lechner GmbH. The vessel may be sealed by a valve having a cup and discharge port, particularly of the type CA38F/39F ex Rexam Dispenser, Portsmouth UK, although valves having a gasket able to withstand steam sterilisation would be suitable for producing a sterile product.

We have also found that the rate at which gel is dispensed may be altered by altering the applicator nozzle size. Thus it is possible to change the applicator in order to get fast release or slow release of the gel which may be important for some wounds.

The invention is illustrated by the following non-limiting examples.

### Example 1

| Gel composition | % by weight |
|---|---|
| Pectin | 0.1% |
| Sodium carboxymethyl cellulose | 3.4% |
| Propylene glycol | 15.0% |
| Purified water | 81.5% |

Pectin (0.2g) was added to purified water (163.0g) in a beaker and heated to 50- 60° C. with constant stirring until the pectin dissolved. Propylene glycol (30.0g) was added and sodium carboxymethyl cellulose (6.8g) was gradually added with constant mixing. A hydrocolloid gel (200g) was produced.

### Example 2

The gel from example 1 was used to fill the inner container of a barrier aerosol vessel and a valve having a cup and discharge port applied to seal the vessel. The filled container was terminally steam sterilised for 30 minutes at 121C. The vessel was then removed to a clean room and an applicator fitted to the valve and the outer container gassed to pressurise the gel.

### Example 3

The barrier aerosol vessel containing gel prepared as in Example 2 was subjected to a microbial challenge. A mixed microbial suspension (S.aureus, E.coli and C. albicans-all typical wound bacteria) was prepared at a concentration of 1×10^{5/}ml and inoculated into the first 2cms of gel contained in the nozzle of the vessel. The inoculated canned gel was then left to stand at room temperature for a period of 7 days and then sampled. This mimics clinical use. After sampling the gel was re-inoculated with the microbial suspension and sampled after a further 7 days. Sampling was achieved by 10 fold dilution plating out appropriate dilutions onto pre-dried TSA plates. The plates were incubated at 35C for 24/48 hours prior to counting.

The results of the assay demonstrated a 5 log reduction in each of the three challenge organisms over days 0-7 and 7-14. These results show that micro-organisms do not proliferate in the gel contained in the barrier vessel. This makes the combination of gel and barrier vessel suitable for a multi-dose sterile product.

1. A barrier aerosol vessel containing a wound gel for the treatment of wounds.

2. A vessel as claimed in claim 1 wherein the gel comprises a hydrocolloid.

3. A vessel as claimed in claim 1 or claim 2 wherein the gel comprises a natural gelling agent.

4. A vessel as claimed in any preceding claim wherein the gel comprises a glycol.

5. A vessel as claimed in any preceding claim wherein the gel comprises:
(a) from about 0.05w to 10% by weight of a natural gelling agent;
(b) from about 1.0% to 10% by weight of a hydrocolloid;
(c) from about 5.0% to 30.0% by weight of an alkylene glycol and
(d) at least 500 by weight of water.

6. A vessel as claimed in any preceding claim wherein the gel is sterile.

7. A vessel as claimed in any preceding claim wherein the vessel contains multiple doses of wound gel.

8. Method of making a barrier aerosol vessel comprising wound gel the method comprising the steps of:
(I) filling an inner container with gel, said inner container being contained within an outer casing container;
(ii) sealing the inner container with an opening valve; and
(iii) introducing a pressure medium between the inner container and the outer casing container.

9. Method of making a barrier aerosol vessel comprising wound gel the method comprising the steps of:
(i) filling an inner container with non-sterile gel, said inner container being contained within an outer casing container;
(ii) sealing the inner container with an opening valve;
(iii) sterilising the vessel and gel contained within it; and
(iv) introducing a pressure medium between the inner container and the outer casing container.

10. A multiple dose, sterile wound gel contained within an aerosol vessel.

11. Use of a barrier aerosol vessel containing a wound gel for the preparation of a wound care product for use in the treatment of wounds.

12. Use of a barrier aerosol vessel containing a wound gel for the preparation of a wound care product for use in the treatment of sinus wounds.
